(19) United States
(12) Patent Application Publication
(10) Pub. No.: US 2018/0200698 A1
(43) Pub. Date: Jul. 19, 2018

(54) CATALYST AND METHODS FOR THE ISOMERISATION OF OLEFINS FROM OLEFIN-CONTAINING HYDROCARBON MIXTURES HAVING 4-20 C-ATOMS

(71) Applicant: BASF SE, Ludwigshafen am Rhein (DE)

(72) Inventors: Stefan ISELBORN, Frankenthal (DE); Andreas Jörg UFER, Mannheim (DE)

(21) Appl. No.: 15/744,449

(22) PCT Filed: Jul. 8, 2016
(86) PCT No.: PCT/EP2016/066224

§ 371 Date: Jan. 12, 2018

(30) Foreign Application Priority Data
Jul. 13, 2015 (EP) 15176448.7

(51) Int. Cl. B01J 23/44; C07C 5/25; B01J 21/04; B01J 35/08; B01J 35/02; B01J 35/00; B01J 35/10; B01J 37/02; B01J 37/18; B01J 38/10

(52) U.S. Cl. CPC B01J 23/44; C07C 5/2556; B01J 21/04; B01J 35/08; B01J 35/023; B01J 35/008; C07C 2523/44; B01J 35/1019; B01J 37/0201; B01J 37/0236; B01J 37/18; B01J 38/10; C07C 2521/04; B01J 35/1014

(57) ABSTRACT

The present invention provides a catalyst comprising alumina as carrier material and palladium or platinum as active component, obtainable by
a) impregnating an alumina carrier with a solution comprising at least one salt of the active component palladium or platinum,
b) drying the catalyst thus obtained,
c) treating the catalyst thus obtained with hydrogen, or a mixture of hydrogen and at least one inert gas, for a period of 1 to 24 hours at a temperature of 30 to 200° C., and
d) thereafter keeping the catalyst thus reduced in the presence of hydrogen, or a mixture of hydrogen and at least one inert gas, for a period of 1 hour to 10 days at a temperature of 10 to 100° C.

The catalyst provided by the invention is useful in processes for isomerization of olefins from olefin-containing hydrocarbonaceous mixtures having 4 to 20 carbon atoms at temperatures of 10 to 150° C. and pressures of 1 to 35 bar.

The present invention relates to palladium- or platinum-containing catalysts on an alumina carrier and processes for isomerization of olefins on olefin-containing hydrocarbonaceous mixtures of 4 to 20 carbon atoms, in particular to a process for isomerizing 1-butene to 2-butene by using these catalysts.

Linear alpha-olefins, in particular linear alpha-olefins having 4 to 8 carbon atoms, are obtained in petrochemical processes such as catalytic or thermal cracking, pyrolysis, dimerizations, oligomerization or Fischer-Tropsch syntheses or as by-products of chemical processes such as raffinate from methyl tert-butyl ether production or butadiene processes. The alpha-olefins thus obtained, which contain a terminal C—C double bond, have to undergo an isomerizing rearrangement into the thermodynamically favored linear internal olefins of the same number of carbon atoms for further processing into other products. These internal olefins of 4 to 8 carbon atoms may, for example, be introduced into a metathesis reaction to produce other olefins; are alkylated to produce gasoline; or are converted into the desired products in other reactions, for example electrophilic additions, dimerizations, oligomerizations and (co)polymerizations.

Numerous processes for isomerizing terminal into internal double bonds in olefins are known. Isomerization reactions of this type may be carried out with hydrogen, as hydroisomerization, and without hydrogen. In either case, however, the right catalyst has to be used. Oligomerization and skeletal isomerization occur as secondary reactions in the absence of hydrogen. Hydroisomerization may result in the hydrogenation of the double bond to obtain saturated products. Economically viable practice of hydroisomerization with minimization of double bond hydrogenation requires optimization and policing of reaction conditions.

Processes for isomerization of alpha-olefins are already known in principle.

EP 0 841 090 A2 describes a catalyst used for isomerization of 3-buten-1-ol compounds. It is a fixed-bed catalyst comprising palladium and selenium or tellurium or a mixture of selenium and tellurium on a silica carrier and having a BET surface area of 80 to 380 m²/g and a pore volume of 0.6 to 0.95 cm³/g in the pore diameter range from 3 nm to 300 μm, while 80 to 95% of the pore volume is in the pore diameter range from 10 to 100 nm. Said fixed-bed catalyst is prepared by a silica carrier being impregnated with a solution of a palladium compound and a selenium or tellurium compound or of a mixture of a selenium compound and a tellurium compound, dried and reduced in the presence of hydrogen.

US 2006/0235254 A1 discloses a process for isomerizing 1-butene into 2-butene in the presence of a catalyst and hydrogen. The purpose of this process is to minimize as far as possible the amount of butane formed. The catalyst comprises palladium, platinum or nickel on alumina and is optionally sulfurized before use.

EP 0 636 677 B1 discloses a process for isomerization of external olefins into internal olefins. This process is carried out in the presence of a catalyst comprising palladium on a carrier material. The catalyst may optionally comprise from 0.05 to 10% of sulfur.

U.S. Pat. No. 3,531,545 discloses a process for isomerization of olefins into 2-olefins. The catalyst used therein comprises a noble metal on alumina. The isomerization is optionally carried out in the presence of a sulfur-containing compound.

Disadvantages of existing processes include low yields, for example as a result of secondary reactions, such as branching, low selectivity and high catalyst costs. Existing processes are further observed to be insufficiently active, i.e., to insufficiently isomerize the starting compounds into the desired products, and allow excessive hydrogenation into saturated compounds.

The problem addressed by the present invention was therefore that of providing an improved catalyst and process for isomerization, in particular hydroisomerization, of olefins from olefin-containing hydrocarbonaceous mixtures of 4 to 20 carbon atoms, in particular of linear alpha-olefins having 4 to 8 carbon atoms, in an improved yield and selectivity for the desired products in combination with a reduced formation of undesired by-products, for example saturated compounds.

The problem is solved by a catalyst comprising alumina as carrier material and palladium or platinum as active component, obtainable by
a) impregnating an alumina carrier with a solution comprising at least one salt of the active component palladium or platinum,
b) drying the catalyst thus obtained,
c) treating the catalyst thus obtained with hydrogen, or a mixture of hydrogen and at least one inert gas, for a period of 1 to 24 hours at a temperature of 30 to 200° C., and
d) thereafter keeping the catalyst thus reduced in the presence of hydrogen for a period of 1 hour to 10 days at a temperature of 10 to 100° C.

The metals to be deposited on the carrier as per process step a)—palladium or platinum—may be applied to the carrier using any known method, for example by coating from the gas phase (chemical or physical vapor deposition) or by impregnating the carrier material with a solution comprising the compounds and/or substances to be deposited.

The preferred method is to impregnate with a solution of their salts (or mixtures thereof) which will, in the further course of the process for preparing the catalyst, convert into the substances to be deposited. These metal salts may be deposited singly and/or in portions in two or more process steps or conjointly and wholly in one process step.

Useful metal salts include particularly metal salts readily convertible by calcination into the corresponding oxides, examples being hydroxides, carbonates, chlorides, nitrates, nitrites, acetates and formates. Some of these metal salt solutions are necessarily acidic because of the anions used. Neutral solutions are preferably rendered acidic with, for example, mineral acids prior to the impregnating step.

The carrier is generally impregnated with a solution of salts of the components to be deposited by the incipient wetness method wherein the volume of the solution is determined such that the pore volume of the carrier takes up virtually the entire solution. The concentration of the salts in the solution is determined such that the components to be deposited are present on the catalyst in the desired concentration after the impregnated carrier has been converted into the final catalyst. The salts are chosen such that they do not leave behind any residues to disrupt the process of preparing the catalyst or its later use.

The catalyst of the present invention is preferably prepared by a single step of impregnating the carrier according to the incipient wetness method, for example i) with a nitric acid solution of the nitrates of the metals to be deposited or ii) with a hydrochloric acid solution of the chlorides of the metals to be deposited, in particular with a hydrochloric acid solution of palladium chloride. The concentration of the nitric acid used in case i) is at least sufficient to ensure a solution that is clear. In general, the solution pH is not more than 5, preferably not more than 2 and more preferably not more than 1. When the carrier is impregnated by using a hydrochloric acid solution of palladium chloride, this solution is first neutralized and it is the resultant palladium hydroxide which is applied to the carrier. Subsequently, the impregnated carrier is washed.

After impregnation, the impregnated carrier is dried in process step b) in a conventional manner, generally at a temperature above 60° C., preferably above 80° C. and more preferably above 100° C., for example at a temperature in the range from 120° C. to 300° C. Drying is continued until substantially all the water present in the impregnated carrier has escaped, which will generally be the case after some hours. Drying periods typically range from one to 30 hours and depend on the setting for the drying temperature, in that a higher temperature shortens the drying time. Drying may further be hastened by applying a negative pressure.

The catalyst thus obtained is then activated in the subsequent steps of the process, process steps c) and d), the first of which, process step c), involves a reducing reaction being carried out for a period of 1 to 24 hours, preferably 3 to hours, more preferably 6 to 14 hours at a temperature of 30 to 200° C., preferably 50 to 180° C., more preferably 60 to 130° C. by treatment with hydrogen or a mixture of hydrogen and at least one inert gas, for example noble gases such as helium, neon or argon, nitrogen, carbon dioxide and/or low alkanes, such as methane, ethane, propane and/or butane. Nitrogen is one preferred inert gas. The concentration of such inert gases in the hydrogen is preferably less than 30% by volume.

Process step d) thereafter comprises keeping the catalyst thus reduced in the presence of hydrogen, or a mixture of hydrogen and at least one of the inert gases referred to above, for a period of 1 hour to 10 days, preferably 6 hours to 8 days, more preferably 1 to 7 days, yet more preferably 3 to 6 days at a temperature of 10 to 100° C., preferably 20 to 80° C., more preferably 25 to 60° C.

A preferred embodiment of the catalyst according to the present invention comprises alumina as carrier material and palladium or platinum as active component, obtainable by
a) impregnating an alumina carrier with a solution comprising at least one salt of the active component palladium or platinum,
b) drying the catalyst thus obtained,
c) treating the catalyst thus obtained with hydrogen, or a mixture of hydrogen and at least one inert gas, preferably nitrogen, for a period of 1 to 24 hours, preferably 3 to 20 hours, more preferably 6 to 14 hours at a temperature of 30 to 200° C., preferably 50 to 180° C., more preferably 60 to 130° C., and
d) thereafter keeping the catalyst thus reduced in the presence of hydrogen, or a mixture of hydrogen and at least one inert gas preferably nitrogen, for a period of 1 hour to 10 days, preferably 6 hours to 8 days, more preferably 1 to 7 days, yet more preferably 3 to 6 days at a temperature of to 100° C., preferably 20 to 80° C., more preferably 25 to 60° C.

In a further preferred embodiment of the catalyst according to the present invention, the catalyst is subjected after process step c) and before process step d) to an additional process step c₁) of being maintained in the hydrogen atmosphere for a further 1 to 16 hours, preferably 2 to 12 hours, more preferably 4 to 10 hours, at a temperature of 10 to 100° C., preferably 20 to 80° C., with the proviso that the temperatures in process steps c), c₁) and d) differ from each other.

In a further embodiment of the catalyst according to the present invention, the catalyst is calcined after drying in process step b) and before the hydrogen treatment in process step c).

This calcination is preferable when the nitrates of the metals to be deposited are used, and is essentially designed to convert the impregnant salts into the components to be deposited, or into precursors thereof, and hence differs from the calcination described hereinbelow, which is designed to produce the carrier material and the carrier structure. In the case of metal nitrates as impregnants, this calcination essentially decomposes the nitrates into metals and/or metal oxides, which remain in the catalyst, and nitrous gases, which escape.

The calcination temperature is generally in the range from 250° C. to 900° C., preferably in the range from 280° C. to 800° C. and more preferably in the range from 300° C. to 700° C. The calcination period is generally between 0.5 and hours, preferably between 0.5 and 10 hours and more preferably between 0.5 and 5 hours. The calcination is carried out in a customary oven, for example in a rotary tube oven, in a belt calciner or in a chamber over. The calcining step may be carried out directly following the drying step, i.e., without an intervening period for the impregnated and dried carrier to cool down.

A further preferred embodiment of the catalyst according to the present invention comprises alumina as carrier material and palladium or platinum as active component, obtainable by
a) impregnating an alumina carrier with a solution comprising at least one salt of the active component palladium or platinum,
b) drying the catalyst thus obtained,
c) treating the catalyst thus obtained with hydrogen, or a mixture of hydrogen and at least one inert gas, preferably nitrogen, for a period of 1 to 24 hours, preferably 3 to 20 hours, more preferably 6 to 14 hours at a temperature of 30 to 200° C., preferably 50 to 180° C., more preferably 60 to 130° C., and
d) thereafter keeping the catalyst thus reduced in the presence of hydrogen, or a mixture of hydrogen and at least one inert gas preferably nitrogen, for a period of 1 hour to 10 days, preferably 6 hours to 8 days, more preferably 1 to 7 days, yet more preferably 3 to 6 days at a temperature of 10 to 100° C., preferably 20 to 80° C., more preferably 25 to 60° C.,
   and then contacting the catalyst with atmospheric oxygen.

The step of treating the catalyst of the present invention with atmospheric oxygen may be effected in a conventional manner, for example by replacing the hydrogen and/or hydrogen/inert gas mixture in process step d) with a nitrogen/atmospheric oxygen mixture (at a nitrogen to air ratio of from 5:1 to 1:5, preferably from 2:1 to 1:2, more preferably equal to 1:1) at temperatures in the range from 20° C. to 35° C. and packaging the catalyst in the presence of this nitrogen/atmospheric oxygen mixture.

In another possible form of the step of contacting with atmospheric oxygen, the catalyst after process step d) is first flushed with nitrogen and then packaged in the presence of atmospheric oxygen without inertization.

The shape of the carrier material may differ in different versions of the invention. When the process is practiced as a suspension process, the carrier material used to prepare the catalysts of the present invention will typically take the form of a finely divided powder. The particle size of the powder is preferably in the range from 1 to 200 μm, in particular from 1 to 100 μm. When the catalyst is used in the form of a fixed bed, it is customary to use shapes molded from the carrier material, for example by extrusion, strand-pressing or tableting, examples being spheres, tablets, cylinders, strands, rings, i.e., hollow cylinders, stars and the like. Dimensions for these molded shapes range typically from 1 mm to 25 mm. Catalyst strands having strand diameters of 1.5 to 5 mm and strand lengths of 2 to 25 mm find frequent use.

It is particularly preferred to use the alumina carrier material for preparing the catalyst of the present invention in the form of molded spheres.

The molded spheres are generally from 1 to 6 mm, preferably from 2 to 5.5 mm and more preferably from 3 to 5 mm in diameter.

The molded shapes, in particular the molded spheres, preferably have with advantage a (side) compressive strength of >40 newtons (N), preferably of >50 N, more preferably of >60 N, more preferably of >70 N, e.g., in the range from 60 to 90 N.

To determine (side) compressive strength of a molded catalyst shape, two parallel plates each exerted increasing force on, for example, the catalyst sphere therebetween or, for example, on the shell surface of the catalyst tablet therebetween, until fracture occurred. The force registered at the point of fracture is the (side) compressive force. The determination was carried out on a tester from Zwick of Ulm, having a fixed rotatable plate and a freely movable vertical punch which pressed the molded shape against the fixed rotatable plate. The freely movable punch was connected to a load cell for recording the force. The instrument was controlled by a computer which registered and evaluated the measured values. (Side) compressive strength was measured on 25 defect-free (i.e., crack-free and, where applicable, without scuffed edges) molded shapes taken from a thoroughly mixed sample of the catalyst and subsequently averaged.

The molded shapes in the form of extrudates preferably have with advantage a cutting hardness of >30 newtons (N), particularly of >40 N, further particularly of >50 N, e.g., in the range from 45 to 70 N.

Cutting hardnesses were measured on an apparatus from Zwick (of the type: BZ2.5/TS1 S; initial force: 0.5 N, initial force speed: 10 mm/min; sinking speed: 1.6 mm/min) and are the average values of 15 catalyst strands measured in each case, Cutting hardness was determined in detail as follows: Extrudates were subjected to a blade 0.3 mm in thickness subjected to increasing force until the extrudate had severed. The force needed for this is the cutting hardness in N (newton). The determination was carried out on a tester from Zwick of Ulm, having a fixed rotatable plate and a freely movable vertical punch having an incorporated blade of 0.3 mm thickness. The movable punch with the blade was connected to a load cell for recording the force and during measurement pressed against the fixed rotatable plate on which the extrudate to be measured lay. The tester was controlled by a computer which registered and evaluated the measured values. Cutting hardness was measured on 15 straight, ideally crack-free extrudates having an average length of 2 to 3 times their diameter, which were taken from a thoroughly mixed sample of the catalyst, and subsequently averaged.

Electron microscopy (SEM or TEM) has further shown that the catalyst of the present invention is an eggshell catalyst. The concentration of the active component within a catalyst particle decreases from out to in, while there is a palladium or platinum layer at the particle surface. In preferred cases, crystalline palladium or platinum is detectable in the eggshell by selected area diffraction (SAD) and x-ray diffraction (XRD).

The active component resides essentially in an outer near-surface layer of the carrier. Generally more than 80 wt %, preferably more than 90 wt % and more preferably more than 95 wt % of the active component resides in a layer not more than 2000 micrometers in thickness and bounded by the geometric surface of the catalyst particle. This is preferably not more than 1000 micrometers in thickness, more preferably not more than 300 micrometers in thickness and most preferably in the range from 150 to 30 micrometers.

Another feature of the catalyst according to the present invention is that the active component is present therein in a highly disperse state.

The dispersity of the active component in the catalyst is on average preferably in the range from 20 to 60%, in particular in the range from 30 to 50% (all measured via CO sorption to DIN 66136-3).

In a further particularly preferred embodiment of the catalyst described at the outset, the active component used is palladium in an amount of 0.05 to 2.0 wt %, preferably 0.1 to 1.0 wt %, more preferably 0.2 to 0.8 wt %, most preferably 0.25 to 0.5 wt %, based on total catalyst weight.

In a further particularly preferred embodiment of the palladium-containing catalyst described at the outset, the solution used to impregnate the alumina carrier comprises palladium chloride and/or palladium hydroxide.

The alumina carrier of the catalyst according to the present invention is preferably a mixture of δ-, θ- and α-alumina, more preferably of δ-, θ-, κ- and α-alumina. The carrier may further comprise other added substances to a certain degree as well as unavoidable impurities. It may for example comprise other inorganic oxides such as oxides of metals of groups IIA, IIIB, IVB, IIIA and IVA of the periodic table, in particular silica, titania, zirconia, zinc oxide, magnesia, sodium oxide and calcium oxide. The maximum presence in the carrier of such oxides, other than alumina, depends on the oxide actually present, but is in an individual case quantifiable from the x-ray diffraction diagram of the catalyst, since a change in structure is accompanied by a significant change in the x-ray diffraction diagram. The presence of such oxides, other than alumina, is generally below 50 wt %, preferably below 30 wt %, more preferably below 10 wt %. The purity of the alumina is preferably above 99%.

To prepare the carrier, a suitable aluminum-containing raw material, preferably gibbsite, is peptized with a peptizing agent, such as water, a dilute acid or a dilute base. The acid used is, for example, a mineral acid, for instance nitric acid, or an organic acid, for instance formic acid. The base used is preferably an inorganic base, for instance ammonia. The acid or base is generally dissolved in water. Water and dilute aqueous nitric acid are the preferred peptizing agents. The concentration of the nonaqueous fraction in the peptizing agent is generally from 0 to 10 wt %, preferably from 0 to 7 wt %, more preferably from 0 to 5 wt %. Peptization is continued until the material is efficiently moldable. The material is then molded into the desired carrier shapes via customary methods, for example by strand-pressing, extrusion, tableting or agglomeration. Any known method of molding is suitable. If necessary or advantageous, customary added substances may be used. Examples of such added substances are extrusion or tableting assistants, such as polyglycols or graphite.

It is further possible for the raw carrier material prior to molding to be admixed with added substances known to act as burnout materials that influence the pore structure of the carrier after calcination, examples being polymers, fibrous materials, natural burnout materials, such as nutshell meals, or other customary added substances. Preference is given to the use of boehmite in a particle size distribution and adding burnout materials that leads to a pore radius distribution for the final carrier where 50-90% by volume of the entire pore volume is in the form of pores having an average diameter in the range from 0.01 to 0.1 μm and from 10% to 50% by volume of the entire pore volume is in the form of pores having an average diameter in the range from 0.1 to 1 μm. The required measures for this are known per se to a person skilled in the art.

After molding, the molded shapes are dried in a conventional manner, generally at a temperature above 60° C., preferably above 80° C., more preferably above 100° C., in particular at a temperature in the range from 120 to 300° C. Drying is continued until substantially all the water present in the molded shapes has escaped therefrom, which will generally be the case after some hours. Customary drying periods range from 1 to 30 hours and depend on the setting of the drying temperature, in that a higher temperature shortens the drying time. Drying may be further hastened by applying a negative pressure.

After drying, the molded shapes are converted into the final carrier by calcination. The calcination temperature is generally in the range from 900 to 1150° C., preferably in the range from 1000 to 1120° C., more preferably in the range from 1050 to 1100° C. The calcination period is generally between 0.5 and 5 hours, preferably between 1 and 4 hours, more preferably between 1.5 and 3 hours. Calcination is carried out in a customary oven, for example in a rotary oven, in a tunnel oven, in a belt calciner or in a chamber oven. The calcining step may be carried out directly following the drying step, i.e., without an intervening period for the molded shapes to cool down.

The alumina carrier thus obtained has a specific surface area (BET, Brunauer-Emmet-Teller, determined as per DIN 66131 by nitrogen adsorption at 77 K) of 20 to 200 m²/g, preferably of 30 to 100 m²/g, more preferably of 35 to 90 m²/g. Surface area may be varied by known methods, in particular using more finely divided or coarser starting materials, calcination period and calcination temperature.

After calcination, the active material and optionally further added-substance materials are deposited as already described at the outset on the carrier thus obtained.

The invention also provides a process for preparing abovementioned catalyst by
a) impregnating an alumina carrier with a solution comprising at least one salt of the active component palladium or platinum,
b) drying the catalyst thus obtained,
c) treating the catalyst thus obtained with hydrogen, or a mixture of hydrogen and at least one inert gas, for a period of 1 to 24 hours at a temperature of 30 to 200° C. after drying, and
d) keeping the catalyst thus reduced in the presence of hydrogen, or a mixture of hydrogen and at least one inert gas, for a period of 1 hour to 10 days at a temperature of 10 to 100° C.,
   and optionally a subsequent step of contacting the catalyst with atmospheric oxygen.

The process descriptions provided at the outset, including the preferred embodiments, may be referenced for a more particular understanding of process steps a) to d).

In a preferred embodiment of the process according to the present invention, the catalyst is subjected after process step c) and before process step d) to an additional process step c₁) of being maintained in the hydrogen atmosphere for a further 1 to 10 hours at a temperature of 10 to 100° C. with the proviso that the temperatures in process steps c), c₁) and d) differ from each other.

In a further preferred embodiment of the process according to the present invention, the solution used to impregnate the alumina carrier in process step a) comprises one or more salts of palladium.

In a further preferred embodiment of the process according to the present invention, the catalyst is calcined after drying in process step b) and before the hydrogen treatment in process step c).

In a further preferred embodiment of the process according to the present invention, the alumina carrier used in step a) is obtained by a₁) treating an aluminum-containing raw material with water, a dilute acid or a dilute base, a₂) molding shapes, a₃) drying the molded shapes, and a₁) calcining the dried molded shapes.

In a further preferred embodiment of the process according to the present invention, the aluminum-containing raw material used in process step a₁) comprises gibbsite.

In a further preferred embodiment of the process according to the present invention, the alumina carrier is subjected to a hydrothermal treatment in an autoclave prior to said drying step a₃).

The present invention further provides a process for activating a catalyst comprising alumina as carrier material and palladium or platinum as active component, which process comprises
c) treating the catalyst with hydrogen, or a mixture of hydrogen and at least one inert gas, for a period of 1 to 24 hours at a temperature of 30 to 200° C., and
d) thereafter keeping the catalyst thus reduced in the presence of hydrogen, or a mixture of hydrogen and at least one inert gas, for a period of 1 hour to 10 days at a temperature of 10 to 100° C.

In a preferred embodiment of the process, said catalyst is obtainable by
a) impregnating an alumina carrier with a solution comprising at least one salt of the active component palladium or platinum, and
b) drying the catalyst thus obtained.

In a further preferred embodiment of the process, the catalyst is brought into contact with atmospheric oxygen following the hydrogen treatment in process step d).

The process descriptions provided at the outset, including the preferred embodiments, may be referenced for a more particular understanding of process steps a) to d).

The addressed problem referred to at the outset was further solved by a process for isomerizing olefins from olefin-containing hydrocarbonaceous mixtures having 4 to 20 carbon atoms, preferably linear aliphatic alpha-olefins from olefin-containing hydrocarbonaceous mixtures having 4 to 8 carbon atoms, more preferably a process for isomerizing 1-butene into 2-butene at temperatures of 10 to 150° C. and pressures of 1 to 35 bar in the presence of the abovementioned catalyst.

### Reactor

The isomerization of the present invention may be carried out in any desired apparatus allowing a continuous mode of operation. The isomerization is preferably carried out in the downflow mode in a tubular reactor containing the fixed-bed catalyst to be used according to the present invention. The tubular reactor in question, preferably in its top half, contains a gas distributor, for example in the form of a filter plate, in the form of a static mixer or in the form of a nozzle. The gas distributor serves to introduce a gas mixture, for example hydrogen/nitrogen, preferably in a uniform manner across the reactor cross section. The compound to be isomerized is initially routed through a heating zone, is mixed with the gas and is routed into the reactor. The space velocity over the catalyst is adjusted such that a preferably 30 to 100%, more preferably 50 to 100%, most preferably 50 to 90%, conversion of the olefin is attained at the point of exit from the reactor.

The process of the present invention is preferably carried out in the presence of hydrogen. In this preferred embodiment, the hydrogen feed rate is adjusted as a function of temperature and total pressure such that a hydrogen partial pressure of 0.1 to 25 bar, preferably 5 to 20 bar, in particular 5 to 12 bar is maintained. Hydrogen passing through into the reactor effluent may, after condensation therefrom of low boilers, be discharged as offgas or be recirculated back into the process. In a further preferred embodiment, the process of the present invention is carried out in the presence of an inert gas, for example nitrogen or methane.

### Process Parameters

The isomerization is preferably carried out at a pressure of 1 to 35 bar absolute, in particular 5 to 25 bar absolute.

The isomerization is generally carried out at temperatures between 10 and 150° C., preferably 30 to 120° C., for example 50 to 100° C. Catalyst space velocity in the present invention is generally operated at from 0.5 to 15 kg/(I_{catalyst}×h), preferably from 1 to 10 kg/I_{catalyst}× h, depending on the starting compound used.

In one preferred embodiment, the isomerization is carried out in the presence of hydrogen. The process of the present invention is therefore preferably carried out in the presence of hydrogen.

In a further preferred embodiment, the isomerization is carried out in the presence of a mixture of hydrogen and an inert gas, preferably methane or nitrogen, wherein the hydrogen used comprises a volume fraction of 80 to 98 mol % relative to the combined amount of hydrogen and methane or nitrogen.

The hydroisomerization process of the present invention may be accompanied by a hydrogenation reaction. This embodiment is preferable when, for example, the reaction mixture further comprises acetylenes, for example butyne and/or vinylacetylene, or dienes, for example butadiene, so these are hydrogenated in the presence of the isomerization catalyst.

The process of the present invention is preferably carried out together with a selective hydrogenation of acetylenes or diolefins. It is preferably 1-butene which is formed in the hydrogenation of acetylenes, for example butyne and/or vinylacetylene, and/or butadiene, and it is then isomerized into 2-butene by the process of the present invention.

### Starting Materials

The olefins to be isomerized according to the present invention may be present as unitary compounds or as a mixture of alpha-olefins having different chainlengths in the range from 4 to 20 carbon atoms, preferably 4 to 8 carbon atoms.

Examples of linear alpha-olefins used as substrates according to the present invention are 1-n-butene, 1-n-pentene, 1-n-hexene, 1-n-heptene and 1-n-octene. The linear alpha-olefins of 4 to 8 carbon atoms that are used for the purposes of the present invention are usable as individual compounds or as a mixture of two or more of the compounds mentioned. Optionally, the reaction mixture may also comprise further organic compounds, for example olefins having two or more double bonds and 4 to 8 carbon atoms, butadiene for example. In the presence of compounds having two or more double bonds, for example acetylenes, all but one are selectively hydrogenated by the preferred presence of hydrogen in a preferred embodiment of the process according to the present invention, to form with preference the corresponding alpha-olefins which are then likewise converted into the corresponding internal olefins in the manner of the present invention.

In general, in the isomerization of the present invention, the double bond in the molecule moves from the 1-position, i.e., the alpha-position, into an internal position. Various internal positions are possible according to the present invention depending on the number of carbon atoms in the substrate. 1-Butene is, for example, isomerizable into 2-butene. 1-Pentene is isomerizable into 2-pentene. 1-Hexene is isomerizable into 2- and/or 3-hexene, etc.

Preferably, in the process of the present invention, the 1-olefins used convert into the corresponding 2-olefins, i.e., the double bond preferably moves from the 1-position into the 2-position in the isomerization of the present invention.

2-Olefins, the formation of which is preferable in the present invention, are obtainable as cis- and/or trans-isomers according to their chainlengths.

It is particularly preferred to isomerize 1-pentene into cis- and/or trans-2-pentene in the process of the present invention. The process of the present invention isomerizes 1-butene into cis- and/or trans-2-butene in a further preferred embodiment wherein the ratio of 2-butene to 1-butene at the point of exit from the isomerization stage is between 3 and 30, preferably between 4 and 25, while the ratio of 2-butene to 1-butene in the stream entering the isomerization stage is between 0.5 and 1.5, preferably between 0.6 and 1.2.

The olefins isomerized according to the present invention, preferably linear 2-olefins, are useful, for example, in the manufacture of propene by metathesis with corresponding further olefins. The internal olefins are further needed for example for the production of gasoline via alkylation or for reactions with other reagents in electrophilic addition reactions such as halogenations, water addition or dimerizations, oligomerizations and polymerizations, for example free-radical reactions of the internal olefins.

Embodiments of the present invention will now be more particularly described by way of example.

### 1. Preparation of Catalysts

### 1.1 Catalyst a (Comparative)

In a mixer, boehmite (Versal 250 from Euro Support, Amsterdam) was moistened with water, intensively mulled until the material was efficiently moldable and then extruded into 3 mm strands. Thereafter, the strands were dried at 120° C. for 2 hours and calcined at 1000° C. for 2 hours. The strands were then impregnated with HNO₃-acidic Pd(NO₃)₂ solution (pH=0.5) by the incipient wetness method, dried at 120° C. for 12 hours and finally calcined at 330° C. for 6 hours. The palladium content of the final catalyst was 0.3 wt %. The specific BET surface area was 70 m²/g.

The catalyst was subsequently treated with hydrogen at 120° C. for 12 hours.

### 1.2 Catalyst B (Comparative)

Molded alpha/theta/kappa-Al₂O₃ spheres 3 mm in diameter and 40 m²/g in specific BET surface area were subjected to an incipient wetness impregnation with a hydrochloric acid PdCl₂ solution neutralized with an NaHCO₃ solution shortly before impregnation. The impregnated carrier was subsequently washed and dried at 150° C. for 12 hours. The palladium content of the final catalyst was 0.25 wt %.

The catalyst was subsequently treated with hydrogen at 120° C. for 12 hours.

### 1.3 Catalyst C (Comparative)

Molded alpha/theta/kappa-Al₂O₃ spheres 3 mm in diameter and 40 m²/g in specific BET surface area were subjected to an incipient wetness impregnation with a hydrochloric acid PdCl₂ solution neutralized with an NaHCO₃ solution shortly before impregnation. The impregnated carrier was subsequently washed and dried at 150° C. for 12 hours. The palladium content of the final catalyst was 0.25 wt %.

### 1.4 Catalyst D (Comparative)

Molded alpha/theta/kappa-Al₂O₃ spheres 3 mm in diameter and 40 m²/g in specific BET surface area were subjected to an incipient wetness impregnation with a hydrochloric acid PdCl₂ solution neutralized with an NaHCO₃ solution shortly before impregnation. The impregnated carrier was subsequently washed and dried at 150° C. for 12 hours. The palladium content of the final catalyst was 0.3 wt %.

The catalyst was subsequently treated with hydrogen at 120° C. for 12 hours.

### 1.5 Catalyst E (Inventive)

Molded alpha/theta/kappa-Al₂O₃ spheres 3 mm in diameter and 40 m²/g in specific BET surface area were subjected to an incipient wetness impregnation with a hydrochloric acid PdCl₂ solution neutralized with an NaHCO₃ solution shortly before impregnation. The impregnated carrier was subsequently washed and dried at 150° C. for 12 hours. The palladium content of the final catalyst was 0.25 wt %.

Thereafter, the catalyst was first treated with hydrogen at 120° C. for 12 hours and then kept in a hydrogen atmosphere for 8 hours at 60° C. and finally for a further 6 days at 30° C.

### 1.6 Catalyst F (Inventive)

Molded alpha/theta/kappa-Al₂O₃ spheres 3 mm in diameter and 40 m²/g in specific BET surface area were subjected to an incipient wetness impregnation with a hydrochloric acid PdCl₂ solution neutralized with an NaHCO₃ solution shortly before impregnation. The impregnated carrier was subsequently washed and dried at 150° C. for 12 hours. The palladium content of the final catalyst was 0.3 wt %.

Thereafter, the catalyst was first treated with hydrogen at 120° C. for 12 hours and then kept in a hydrogen atmosphere for 8 hours at 60° C. and finally for a further 6 days at 30° C.

### 1.7 Catalyst G (Inventive)

Molded alpha/theta/kappa-Al₂O₃ spheres 3 mm in diameter and 40 m²/g in specific BET surface area were subjected to an incipient wetness impregnation with a hydrochloric acid PdCl₂ solution neutralized with an NaHCO₃ solution shortly before impregnation. The impregnated carrier was subsequently washed and dried at 150° C. for 12 hours. The palladium content of the final catalyst was 0.3 wt %.

Thereafter, the catalyst was first treated with hydrogen at 120° C. for 12 hours and then kept in a hydrogen atmosphere for 8 hours at 60° C. and finally for a further 6 days at 30° C.

Subsequently, the catalyst was flushed with nitrogen at 30° C. and then treated for 2 hours with an air-nitrogen mixture in which the proportion of the air stream was incrementally raised over 1 hour to finally 50%.

### 2. 1-Butene (1-Bu) to 2-butene (2-Bu) hydroisomerization Experiments

The 1-butene to 2-butene isomerization experiments were each carried out in a fixed-bed reactor, equipped with recirculator and separator, in the presence of one of the catalysts itemized in the table. The substrate stream (feed) was a raffinate I comprising 0.5-0.6% by volume of the butadiene (BD) and a 0.6 to 0.7 ratio of 2-butene to 1-butene. An additional substrate stream (feed) was a raffinate II without butadiene and having a 0.8 ratio of 2-butene to 1-butene.

The reaction conditions were as follows:

| | |
|---|---|
| whsv [kg/(l × h)] | 8.5 |
| recirculation to feed ratio | 1.9 |
| cross-sectional space velocity [m³/m²/h] | 39 |
| pressure | 8 bar |
| temperature | 60° C. |

In the experiments, the molar H₂/BD ratio in the substrate stream of raffinate I was varied between 2:1 to 3.5:1 (mol/mol) while the other parameters were all left unchanged, so that full conversion of BD was attained in all experiments. The experiment involving raffinate II used an H₂ flow rate in the substrate stream on the order of magnitude of the other experiments.

The composition of the product obtained was evaluated regarding the ratio of 2-butene to 1-butene, the formation of 2-butene and n-butane (n-Bu) and also the 1-butene isomerization.

It transpired that the process of the present invention, involving the use of catalysts E, F or G, was observed to give a distinctly higher isomerization of 1-butene to 2-butene (2-butene to 1-butene ratio from above 4 to above 8) than the comparative tests involving the catalysts A, B, C and D (2-butene to 1-butene ratio between 1 and 3). At the same time, the use of catalyst E, F or G was found to give a comparably low degree of overhydrogenation versus the comparative tests (with catalysts A, B, C and D). The results are tabled hereinbelow.

TABLE
Results of 1-butene to 2-butene hydroisomerization

| | | BD content | 2-Bu/1-Bu | 1-Bu | n-Bu | 2-Bu | 2-Bu |
|---|---|---|---|---|---|---|---|
| | 2-Bu/1-Bu | [vol %] | product | isomerization | formation | yield | formation |
| Catalyst | input feed | input feed | stream | [%]*** | [wt %]**** | [%]***** | [wt %]****** |
| A* | 0.66 | 0.52 | 1.18 | 25.2 | 1.19 | 21.7 | 5.10 |
| B* | 0.65 | 0.51 | 2.76 | 56.3 | 0.95 | 54.0 | 12.74 |
| C* | 0.67 | 0.59 | 2.54 | 53.5 | 1.18 | 50.4 | 12.01 |
| D* | 0.73 | 0.47 | 2.14 | 45.5 | 0.93 | 42.6 | 9.15 |
| E** | 0.65 | 0.63 | 8.20 | 82.3 | 1.23 | 77.6 | 18.47 |
| F** | 0.67 | 0.53 | 4.17 | 68.0 | 1.03 | 64.4 | 14.31 |
| G** | 0.61 | 0.53 | 4.74 | 72.3 | 1.07 | 69.2 | 16.11 |
| E⁺ | 0.82 | 0.00 | 7.96 | 80.1 | 1.30 | 76.5 | 30.11 |

*comparative test on raffinate I
**inventive test on raffinate I
⁺inventive test on raffinate II (without butadiene)
***[1-Bu in hydrogenation feed] − [1-Bu in hydrogenation product]/[1-Bu in hydrogenation feed]
****[n-Bu in hydrogenation product] − [n-Bu in hydrogenation feed]
*****[2-Bu in hydrogenation product] − [2-Bu in hydrogenation feed]/[1-Bu in hydrogenation feed] + [BD in hydrogenation feed]
******[2-Bu in hydrogenation product]/[2-Bu in hydrogenation feed]

1.-29. (canceled)

30. A catalyst comprising alumina as carrier material and palladium or platinum as active component, obtainable by
a) impregnating an alumina carrier with a solution comprising at least one salt of the active component palladium or platinum,
b) drying the catalyst thus obtained,
c) treating the catalyst thus obtained with hydrogen, or a mixture of hydrogen and at least one inert gas, for a period of 1 to 24 hours at a temperature of 30 to 200° C., and
d) thereafter keeping the catalyst thus reduced in the presence of hydrogen, or a mixture of hydrogen and at least one inert gas, for a period of 1 hour to 10 days at a temperature of 10 to 100° C.

31. The catalyst according to claim 30 wherein the catalyst is subjected after process step c) and before process step d) to an additional process step c₁) of being maintained in the hydrogen atmosphere for a further 1 to 10 hours at a temperature of 10 to 100° C. with the proviso that the temperatures in process steps c), c₁) and d) differ from each other.

32. The catalyst according to claim 30 wherein the catalyst is calcined after drying in process step b) and before the hydrogen treatment in process step c).

33. The catalyst according to claim 30 wherein the catalyst is brought into contact with atmospheric oxygen following the hydrogen treatment in process step d).

34. The catalyst according to claim 30 wherein the carrier material used comprises molded spheres of alumina.

35. The catalyst according to claim 34 wherein the molded spheres are from 1 to 6 mm in diameter.

36. The catalyst according to claim 30 wherein the active component is predominantly present therein concentrated into an eggshell at the catalyst surface.

37. The catalyst according to claim 30 wherein the active component is present therein in a highly disperse state.

38. The catalyst according to claim 37 wherein the catalyst has a dispersity in the range from 20 to 60% (as measured via CO sorption to DIN 66136-3).

39. The catalyst according to claim 30 wherein the active component used is palladium in an amount of 0.05 to 2.0 wt %, based on total catalyst weight.

40. The catalyst according to claim 39 wherein the solution used to impregnate the alumina carrier comprises palladium chloride and/or palladium hydroxide.

41. The catalyst according to claim 30 wherein the alumina carrier used has a BET surface area of 20 to 200 m²/g.

42. The process for preparing the catalyst as claimed in claim 30 by
a) impregnating an alumina carrier with a solution comprising at east one salt of the active component palladium or platinum,
b) drying the catalyst thus obtained,
c) treating the catalyst thus obtained with hydrogen, or a mixture of hydrogen and at least one inert gas, for a period of 1 to 24 hours at a temperature of 30 to 200° C. after drying, and
d) keeping the catalyst thus reduced in the presence of hydrogen, or a mixture of hydrogen and at least one inert gas, for a period of 1 hour to 10 days at a temperature of 10 to 100° C.

43. The process for preparing a catalyst according to claim 42 wherein the catalyst is brought into contact with atmospheric oxygen following the hydrogen treatment in process step d).

44. The process for preparing a catalyst according to claim 42 wherein the catalyst is subjected after process step c) and before process step d) to an additional process step c₁) of being maintained in the hydrogen atmosphere for a further 1 to 10 hours at a temperature of 10 to 100° C. with the proviso that the temperatures in process steps c), c₁) and d) differ from each other.

45. The process for preparing a catalyst according to claim 42 wherein the catalyst is calcined after drying in process step b) and before the hydrogen treatment in process step c).

46. The process for preparing a catalyst according to claim 42 wherein the alumina carrier used in step a) is obtained by a₁) treating an aluminum-containing raw material with water, a dilute acid or a dilute base, a₂) molding shapes, a₃) drying the molded shapes, and a₄) calcining the dried molded shapes.

47. The process for preparing a catalyst according to claim 46 wherein the aluminum-containing raw material used in process step a₁) comprises gibbsite.

48. The process for preparing a catalyst according to claim 46 wherein the alumina carrier is subjected to a hydrothermal treatment in an autoclave prior to said drying step a₃).

49. The process for preparing a catalyst according to claim 42 wherein the solution used to impregnate the alumina carrier in process step a) comprises one or more salts of palladium.

50. A process for isomerization of olefins from olefin-containing hydrocarbonaceous mixtures having 4 to 20 carbon atoms wherein the process is conducted at temperatures of 10 to 150° C. and pressures of 1 to 35 bar in the presence of the catalyst as claimed in claim 30.

51. The process according to claim 50 wherein olefins having external double bonds are isomerized into olefins having internal double bonds.

52. The process according to claim 50 wherein 1-butene is isomerized into 2-butene.

53. The process according to claim 50 wherein the isomerization is combined with a selective hydrogenation of diolefins in the olefin-containing hydrocarbonaceous mixtures.

54. The process according to claim 52 wherein the ratio of 2-butene to 1-butene at the point of exit from the isomerization stage is between 3 and 30.

55. The process according to claim 54 wherein the ratio of 2-butene to 1-butene at the point of exit from the isomerization stage is between 4 and 25.

56. A process for activating a catalyst comprising alumina as carrier material and palladium or platinum as active component, which process comprises
c) treating the catalyst with hydrogen, or a mixture of hydrogen and at least one inert gas, for a period of 1 to 24 hours at a temperature of 30 to 200° C., and
d) thereafter keeping the catalyst thus reduced in the presence of hydrogen, or a mixture of hydrogen and at least one inert gas, for a period of 1 hour to 10 days at a temperature of 10 to 100° C.

57. The process for activating a catalyst according to claim 56, wherein said catalyst is obtainable by
a) impregnating an alumina carrier with a solution comprising at least one salt of the active component palladium or platinum, and
b) drying the catalyst thus obtained.

58. The process for activating a catalyst according to claim 56, wherein the catalyst is brought into contact with atmospheric oxygen following the hydrogen treatment in process step d).
